(19) United States
(12) Patent Application Publication
(10) Pub. No.: US 2011/0276033 A1
(43) Pub. Date: Nov. 10, 2011

(54) WIRE GUIDE AND METHOD OF MAKING SAME

(75) Inventors: Thomas Albert Kay, Bloomington, IN (US); James C. Elsesser, Bloomington, IN (US)

(73) Assignee: COOK MEDICAL TECHNOLOGIES LLC, Bloomington, IN (US)

(21) Appl. No.: 13/142,494

(22) PCT Filed: Dec. 29, 2009
(86) PCT No.: PCT/US2009/069674

§ 371 Date: Jun. 28, 2011

Related U.S. Application Data
(60) Provisional application No. 61/141,268, filed on Dec. 30, 2008.

(51) Int. Cl. A61M 25/09; B65H 69/02

(52) U.S. Cl. 604/528; 156/158

(57) ABSTRACT

A method for making a wire guide (62) is provided. The method comprises forming a distal end portion (26) of a proximal core wire (10). The distal end portion has a first variable diameter (32) less than a maximum diameter of the proximal core wire. The first variable diameter is configured to flare distally along at least a partial length of the distal end portion. A proximal end portion (28) of a distal core wire (12) is formed. A proximal end portion has a second variable diameter less than the maximum diameter of the distal core wire. The second variable diameter is configured to flare proximally along at least a partial length of the proximal end portion. The distal and proximal end portions are overmolded with polymeric material to form a joining member that couples the distal and proximal end portions together.

### CROSS REFERENCE TO RELATED APPLICATIONS

This application is related to and claims all available benefit of U.S. provisional patent application 61/141,268 filed Dec. 30, 2009, the entire contents of which are herein incorporated by reference.

### BACKGROUND OF THE INVENTION

1. Field of the Invention

The present invention relates to medical devices. More particularly, the invention relates to wire guides and a method for making wire guides.

2. Background of the Invention

Often elongated, flexible wire guides are used to gain access to specific inner areas of the body. The wire guide may enter the body through a small opening and travel to parts of the body through body channels. For example, wire guides may be passed through the body via peripheral blood vessels, gastrointestinal tract, or the urinary tract. Wire guides are commercial available and are currently used in cardiology, gastroenterology, urology, and radiology. Once in place at a desired location in the body, wire guides are commonly used as guides for the introduction of additional medical instruments, e.g., catheters.

One design challenge for wire guides is that they have sufficient column strength to be pushed through a patient's vascular system or other body lumen without kinking. The wire guide must also be flexible enough to avoid damaging the blood vessel or other body channel through which it is being advanced. Improved strength and enhanced flexibility, however, are two properties which for the most part are diametrically opposed to one another. That is, an increase in one of these properties usually involves a decrease in the other. Accordingly, further improvements and enhancements in the strength and flexibility of wire guides may be desirable.

### BRIEF SUMMARY OF THE INVENTION

In at least one embodiment of the present invention, a method for making a wire guide is provided. The method comprises forming a distal end portion of a proximal core wire. The distal end portion has a first variable diameter that is less than a maximum diameter of the proximal core wire. The first variable diameter is configured to flare distally along at least a partial length of the distal end portion. A proximal end portion of a distal core wire is formed. The proximal end portion has a second variable diameter that is less than the maximum diameter of the distal core wire. The second variable diameter is configured to flare proximally along at least a partial length of the proximal end portion. Polymer material is overmolded onto the distal and proximal end portions to form a joining member that couples the distal and proximal end portions together.

In at least one other embodiment of the present invention, a wire guide is provided. The wire guide comprises a proximal core wire having a distal end portion. The distal end portion has a first variable diameter that is less than a maximum diameter of the proximal core wire. The first variable diameter is configured to flare distally along at least a partial length of the distal end portion. A distal core wire has a proximal end portion that has a second variable diameter that is less than a maximum diameter of the distal core wire. The second variable diameter is configured to flare proximally along at least a partial length of the proximal end portion. A joining member is formed of polymeric material overmolded onto the distal and proximal end portions. The joining member couples the distal and proximal end portions together.

In at least one other embodiment of the present invention, a catheter kit is provided. The catheter kit comprises the wire guide as described in the foregoing paragraph and a guide catheter for insertion into a patient. The wire guide provides the guide catheter a path during insertion into the patient.

Further objects, features, and advantageous of the present invention will become apparent from consideration of the following description and appended claims when taken in conjunction with the accompanying drawings.

### BRIEF DESCRIPTION OF THE DRAWINGS

FIG. 1 is a side view of core wires in accordance with an embodiment of the present invention;

FIG. 2 is a side view of core wires being machined in accordance with one embodiment of the present invention;

FIG. 3 is a side view of core wires being overmolded in accordance with an embodiment of the present invention;

FIG. 4 is a flow chart for a method of making a wire guide in accordance with one example of the present invention;

FIG. 5A is a side view of a wire guide in accordance with an embodiment of the present invention;

FIG. 5B is a sectional view of the wire guide depicted in FIG. 5A;

FIG. 5C is a sectional view of a wire guide in accordance with one embodiment of the present invention;

FIG. 6A is an exploded view of a catheter kit in accordance with one embodiment of the present invention; and

FIG. 6B is a side view of the catheter kit in accordance with another embodiment of the present invention.

### DETAILED DESCRIPTION OF THE INVENTION

Detailed embodiments of the present invention are disclosed herein. It is understood, however, that the disclosed embodiments are merely exemplary of the invention and may be embodied in various and alternative forms. The figures are not necessarily to scale; some figures may be configured to show the details of a particular component. Therefore, specific structural and functional details disclosed herein are not to be interpreted as limiting but merely as a representative basis for the claims and teaching one skilled in the art to practice the present invention.

Examples of the present invention seek to overcome some of the concerns associated with providing a wire guide for guiding various medical devices through a body channel or cavity of a patient while providing sufficient column strength and flexibility to the wire guide so that the wire guide can be pushed through the patients' body without kinking and causing damage to the surrounding body tissue.

Employing the principles of the present invention is, for example, a wire guide, a method for making the wire guide and a catheter kit. The wire guide comprises two separate core wires. The core wires are joined together by a joining member that has been overmolded about one end of each of the core wires. The overmolded ends of the core wires are configured to mechanically anchor to the joining member. By having two discrete core wires joined together, the mechanical properties of each of the core wire can be respectively selected to provide corresponding sections of the wire guide with unique mechanical properties. In one example, the wire guide has a proximal section with relatively high column strength for pushing the wire guide through a patients' body lumen without kinking. In another example, the wire guide has a distal section with relatively high flexibility to avoid damage to a body channel or cavity when the wire guide is advanced therethrough.

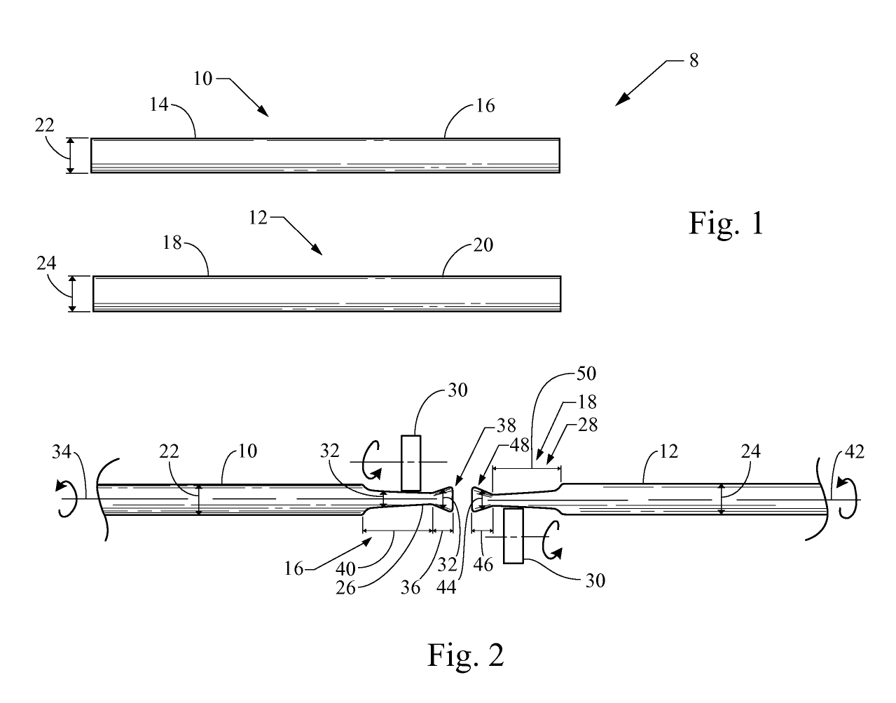

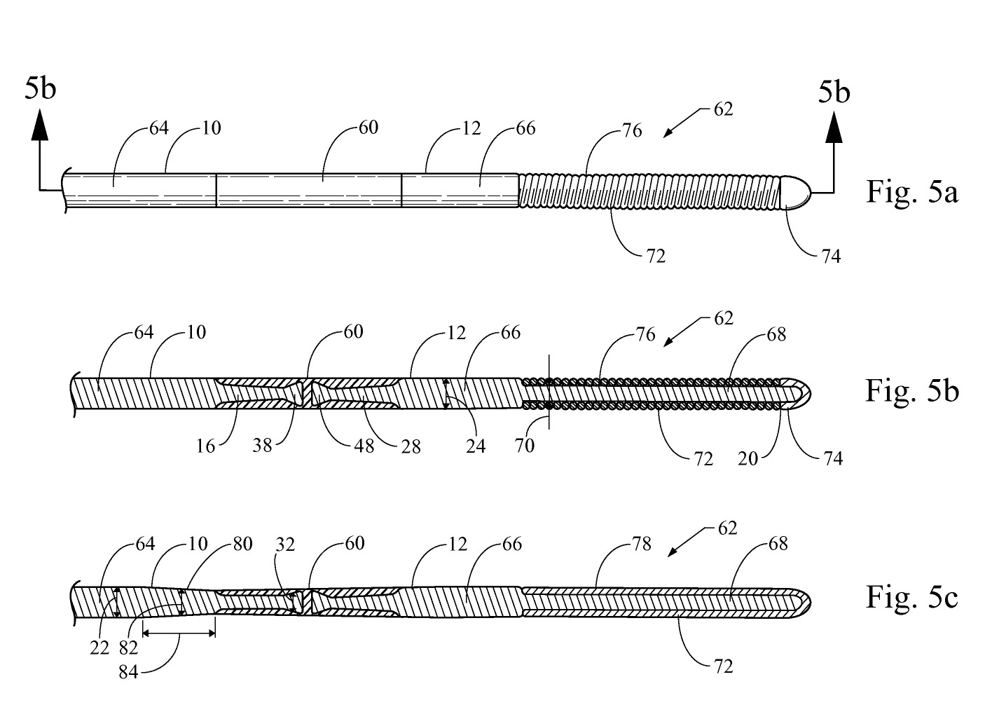

Referring to FIG. 1, a pair of core wires 8 for use in making a wire guide in accordance with at least one embodiment of the present invention is provided. The pair of core wires 8 includes a proximal core wire 10 and a distal core wire 12, which when incorporated into a wire guide (shown in FIGS. 5a-5c) correspond to the proximal and distal sections of the wire guide. The proximal and distal core wires 10 and 12 each have a proximal end 14 and 18 and a distal end 16 and 20.

The core wires 10 and 12 may be made by any suitable wire forming process known to those skilled in the art, such as for example, pultrusion or extrusion of a metal alloy through a mold or die that has a circular opening. The core wires 10 and 12 each have an elongated cylindrical form with a corresponding diameter 22 and 24. Various diameters 22 and 24 for the core wires 10 and 12 are within the scope and spirit of the present invention with many medical procedures preferably having wire guides with a core wire diameter not exceeding about 0.020 inches. In one embodiment, the core wires 10 and 12 have the same or substantially the same (“substantially the same” is hereinafter understood to mean within manufacturing tolerances) diameters 22 and 24. In an alternative embodiment, one of the core wires 10 or 12 has a smaller diameter than the other core wire 10 or 12. For example, the distal core wire 12 may have a smaller diameter 24 than the diameter 22 of the proximal core wire 10 to form a wire guide having a relatively more flexible distal section.

The core wires 10 and 12 may each be comprised of different materials. For example, the proximal core wire 10 may comprise a material that is relatively stiffer and more kink resistant than the material of the distal core wire 12. In one embodiment, the proximal core wire 10 is made from stainless steel and the distal core wire 12 is made from Nitinol.

Referring to FIGS. 2 and 4, an example of a method for making a wire guide is provided. A distal end portion 26 of the proximal core wire 10 is formed at 100. In one embodiment, the distal end portion 26 is formed by removing material from the distal end 16 of the proximal core wire 10 by machining or grinding with a grinding wheel 30 or mill.

The machining or grinding process is preferably capable of machining away material from the core wires 10 and 12 to produce intricate shapes with varying dimensions and geometries. Accordingly, the grinding wheel 30 may be accurately controlled for movement over numerous axes. In one example, this is accomplished by using an automated computer numerically controlled (CNC) multi-axis grinding machine. Preferably, the CNC grinding machine is capable of controlled movement over at least two axes, e.g., the X and Y axis. One such machine is a CAM.2 Profile Grinder manufactured by Glebar Company Inc. The CAM.2 Profile Grinder has direct interface with CAD/CAM and includes a fully integrated multi-axis servo controller. This arrangement may allow for machining of very intricate shapes which have been designed using a CAD based program. In one example, the grinding machine is capable of machining the shape within the core wires 10 and 12 to within several microns of a targeted dimension.

In one embodiment, the distal end 16 of the proximal core wire 10 is machined while the proximal core wire 10 is rotated about its longitudinal axis 34 to form substantially circular cross-sections along the distal end portion 26 with corresponding diameters 32. The diameters 32 are configured to vary (i.e. variable diameter 32) along the distal end portion 26 to define the shape of the distal end portion 26. Because material is removed from the proximal core wire 10 to form the distal end portion 26, the variable diameter 32 is less than the diameter 22 of the proximal core wire 10.

In one embodiment, the variable diameter 32 is configured to flare distally along a distal most length 36 of the distal end portion 16. For example, the variable diameter 32 may vary substantially linearly along the distal length 36 to form a partial cone shape or frustoconical section 38. Alternatively, the variable diameter 32 may vary in a non-linear fashion along the distal length. In another embodiment, the variable diameter 32 is configured to taper distally along a partial length 40 of the distal end portion 16 which is proximal to the distal most length 36. Other suitable configurations and/or arrangements for the distal end portion 16 may also be used without departing from the scope or spirit of the present invention.

The proximal end portion 28 of the distal core wire 12 is formed at 102. Similar to forming of the distal end portion 26, the proximal end portion 28 is formed by removing material from the proximal end 18 of the distal core wire 12 by machining or grinding with the grinding wheel 30 or mill. In one embodiment, the proximal end 18 of the distal core wire 12 is machined while the distal core wire 12 is rotated about its longitudinal axis 42 to form substantially circular cross-sections along the proximal end portion 28 with corresponding diameters 44. The diameters 44 are configured to vary (i.e. variable diameter 44) along the proximal end portion 28 to define the shape of the proximal end portion 28. The variable diameter 44 is less than the diameter 24 of the distal core wire 12.

In one embodiment, the variable diameter 44 is configured to flare proximally along a proximal most length 46 of the proximal end portion 28. For example, the variable diameter 44 may vary substantially linearly along the proximal length 46 to form a partial cone shape or frustoconical section 48. Alternatively, the variable diameter 44 may vary non-linearly. In another embodiment, the variable diameter is configured to taper proximally along a partial length 50 of the proximal end portion 28 which is distal to the proximal most length 46. Other suitable configuration and/or arrangement for the proximal end portion 28 may also be used without departing from the spirit of the present invention.

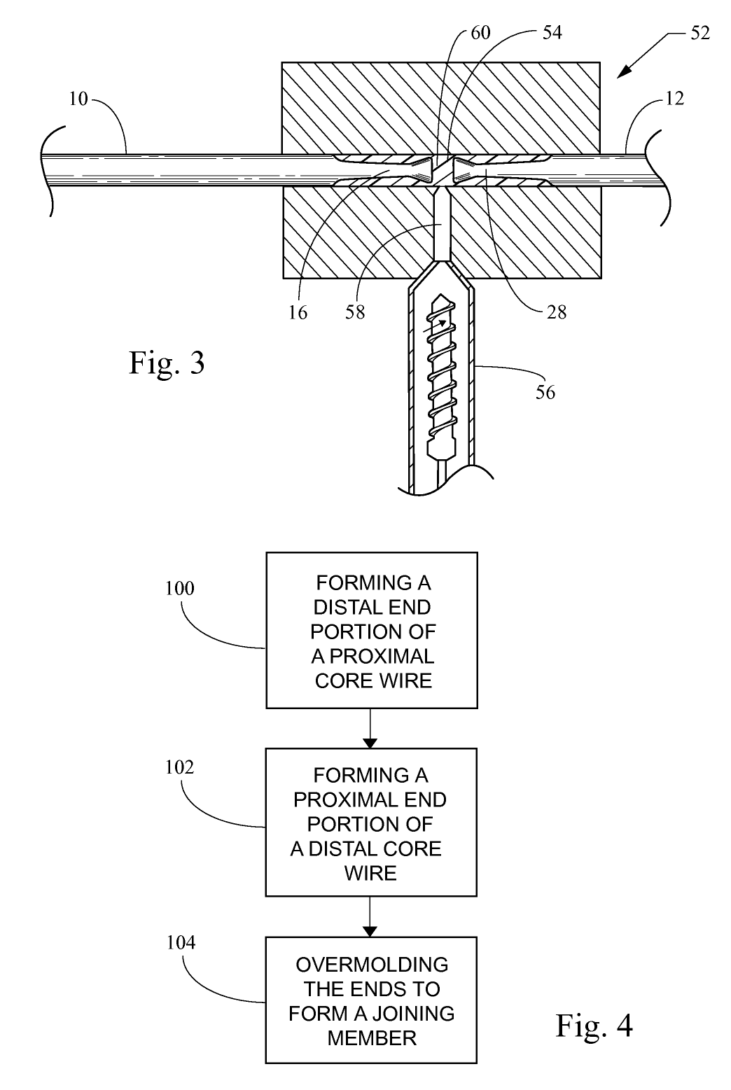

Referring to FIGS. 3 and 4, the distal and proximal end portions 16 and 28 of the proximal and distal core wires 10 and 12 are overmolded at 104. In one embodiment, the proximal and distal core wires 10 and 12 are inserted into a mold 52. As illustrated, the distal and proximal end portions 16 and 28 are longitudinally aligned and spaced apart within the cavity 54 of the mold 52. In one example, the end portions 16 and 28 are spaced apart between about 0.5 to 3.0 mm to facilitate the overmolding process and enhance coupling to the joining member 60 (discussed further below).

In one embodiment, the mold 52 interfaces with an injection molding machine 56 which is configured to deliver molten polymeric material into the cavity 54 of the mold 52 via a runner/gate arrangement 58 formed in the mold 52. The polymeric material may be a thermoplastic or thermoset plastic material. Non-limiting examples of the polymeric material include polyamide, polyester, polyurethane, ABS, polycarbonate, epoxy or blends/mixtures thereof. Other suitable processes and/or systems may also be used for delivering the polymer material into the cavity 54 of the mold 52.

The distal and proximal end portions 16 and 28 are overmolded with the molten polymeric material. That is, the polymeric material is injected into the cavity 54 of the mold 52 around, over, under and/or through the distal and proximal end portions 16 and 28. The mold 52 further facilitates cooling and/or curing of the polymer material to form a solid structure defining a joining member 60. The joining member 60 couples the distal and proximal end portions 16 and 28 together and preferably has a substantially cylindrical form aligned with the proximal and distal core wires 10 and 12 which corresponds to their diameters 22 and 24.

Referring to FIGS. 5A-5C, one embodiment of a wire guide 62 in accordance with the present invention is provided. The wire guide 62 has a proximal section 64 and a distal section 66 that correspond to the proximal and distal core wires 10 and 12 as discussed in the foregoing paragraphs. The frustoconical or otherwise flared sections 38 and 48 of the proximal and distal core wires 10 and 12 mechanically lock the core wires 10 and 12 to the joining member 60. Without being limited by theory, it is believed that by flaring the sections 38 and 48 radially outward over a 360 degree angle, any forces, e.g., tensile, flexural and/or compressive, applied to the wire guide 62 may be more uniformly distributed and transferred to the joining member 60, enhancing the coupling strength between the joining member 60 and the core wires 10 and 12, reducing possible pull-out of one or more of the core wires 10 and 12 from the joining member 60, and providing more uniform bending of the core wires 10 and 12 proximate to the joining member 60.

In one embodiment, the guide wire 62 has a recessed portion 68 formed in the distal core wire 12 distal from the proximal end portion 28. The recessed potion 68, for example, may be formed be removing material from the distal core wire 12 as part of the machining process described in the foregoing paragraphs. The recessed portion 68 has a reduced axial cross-sectional dimension 70 relative to the diameter 24 of the distal core wire 12. This reduced dimension 70 may provide the distal end 20 of the core wire 12 with greater flexibility.

A collar 72 may be positioned adjacent to the distal tip 74 of the wire guide 62 about the recessed portion 68. The distal tip 74, which may be formed for example by soldering or welding the distal end 20, preferably has an outside diameter corresponding to the diameter of the collar 72 so as to retain the collar 72 about the recessed portion 68. The collar 72 may be a coil spring 76 as depicted in FIGS. 5A and 5B or alternatively, may be shrink tubing 78 which has been shrunk to fit about the recessed portion 68 as depicted in FIG. 5C. In one example, the shrink tubing 78 is shrunk by applying heat or ultraviolet radiation to facilitate crystallization and/or cross-linking of the tubing material. Compositions and processing for shrink tubing 78 are generally known in the art and any suitable shrink tubing 78 and associated processing to shrink the tubing 78 may be used.

In one example, the collars 72 attaches to the core wire 12 to form the wire guide 62 by bonding with adhesive. Alternatively, the collar 72 may be attached via soldering or welding. Other suitable means know to those skilled in the art for attaching a collar to a core wire may also be used.

The collar 72 may also include a radio pacifier that is detectible by X-ray and/or fluoroscopic visualization. The radio pacifier may be incorporated directly into the material of the collar 72 or coated thereon. Alternatively, the radio pacifier may be included as part of the adhesive, welding material or solder that is used to attach the collar 72 to the core wire 12.

Referring to FIG. 5C, the proximal core wire 10 of the wire guide 62 in one embodiment has a tapered section 80. The tapered section 80 has a variable diameter 82 that is less than the maximum diameter 22 of the proximal core wire 10 but greater than the variable diameter 32 of the distal end portion 16. The variable diameter 82 is configured to taper distally along a length 84 of the proximal core wire 10 that is proximal to the joining member 60. In one example, the tapered section 80 provides a smooth modulus transition for the wire guide 62 from its stiffer proximal core wire 10 to its more flexible distal core wire 12.

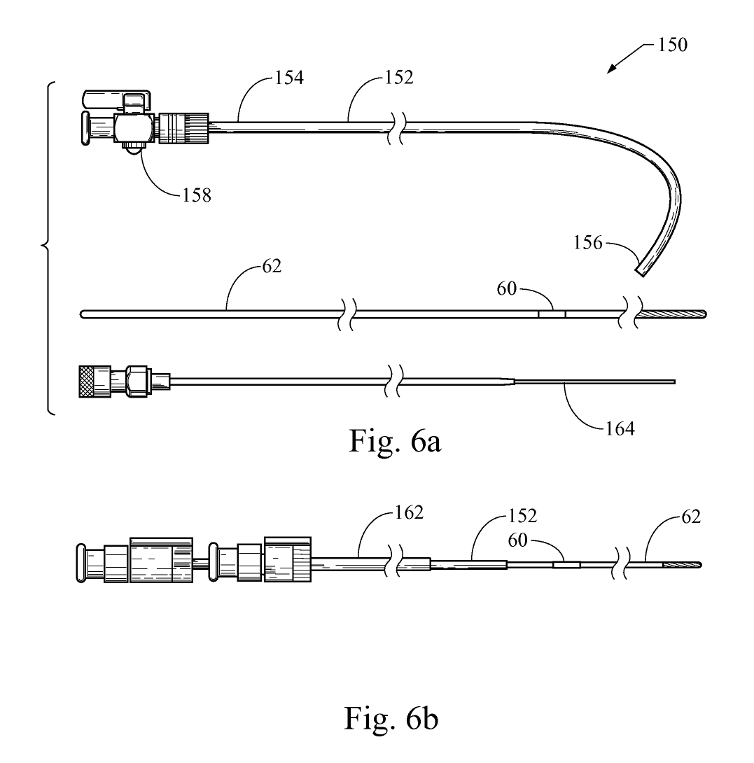

Referring to FIGS. 6A-6B, a catheter kit 150 for a body channel or cavity is provided. As shown, the kit 150 includes a microcatheter 152 preferably made from a soft, flexible material such as silicone or any other suitable material. Generally, the microcatheter 152 has a proximal end 154, a distal end 156, and a plastic adapter or hub 158 to receive a medical device (not shown), e.g., angioplasty balloon, stent, occluding device, etc., to be advanced therethrough. In this embodiment, the inside diameter of the microcatheter 52 may range between 0.014 and 0.027 inch.

The kit 150 further includes the wire guide 62 as discussed in the foregoing paragraphs. The wire guide 62 provides a guide catheter 162 (discussed in more detail below) a path during insertion of the guide catheter 162 within the body channel or cavity. The size of the wire guide 62 is based on the inside diameter of the guide catheter 162.

The guide catheter 162 or sheath is typically made from polytetrafluoroethylene (PTFE) and is for percutaneously introducing the microcatheter 152 into the body of the patient. Of course, any other suitable material may be used without falling beyond the scope or spirit of the present invention. The guide catheter 162 may have a size of between about 4-French to 8-French and allows the microcatheter 152 to be inserted therethrough to a desired location in the body channel or cavity. The guide catheter 162 receives the microcatheter 152 and provides stability of the microcatheter 152 at a desired location within the body. For example, the guide catheter 152 may stay stationary within a common visceral artery, e.g., a common hepatic artery, and adds stability to the microcatheter 152 as the microcatheter 152 is advanced through the guide catheter 162 to a desired point in a connecting artery, e.g., the left or right hepatic artery.

When the distal end 156 of the microcatheter 152 is at the desired point in the body, the medical device may be loaded at the proximal end 154 of the microcatheter 152 and is advanced through the microcatheter 152 for deployment through the distal end 156. In one embodiment, a push wire 164 is used to mechanically advance or push the medical device through the microcatheter 152. The size of the push wire 164 depends on the diameter of the microcatheter 152.

It is to be understood that the catheter kit 150 described above is merely one example of a kit that may be used with the wire guide 62. Of course, other kits, assemblies, and systems may be used with the wire guide 62 without falling beyond the scope or spirit of the present invention.

As a person skilled in the art will readily appreciate, the above description is meant as an illustration of the implementation of the principles of this invention. This description is not intended to limit the scope of application of this invention in that the invention is susceptible to modification, variation, and change, without departing from the spirit of this invention, as defined in the following claims.

What we claim is:

1. A method for making a wire guide, the method comprising:
forming a distal end portion of a proximal core wire, the distal end portion having a first variable diameter less than a maximum diameter of the proximal core wire, the first variable diameter is configured to flare distally along at least a partial length of the distal end portion;
forming a proximal end portion of a distal core wire, the proximal end portion having a second variable diameter less than a maximum diameter of the distal core wire, the second variable diameter is configured to flare proximally along at least a partial length of the proximal end portion; and
overmolding the distal and proximal end portions with polymeric material to form a joining member that couples the distal and proximal end portions together.

2. The method according to claim 1 wherein the first variable diameter is configured to flare distally to form a first frustoconical section of the distal end portion, and the second variable diameter is configured to flare proximally to form a second frustoconical section of the proximal end portion.

3. The method according to claim 1 wherein the proximal core wire comprises a first material and the distal core wire comprises a second material different than the first material, the first material being relatively stiffer than the second material.

4. The method according to claim 3 wherein the second material is relatively more kink resistant than the first material.

5. The method according to claim 1 wherein the polymeric material comprises polyamide, polyester, polyurethane, ABS, polycarbonate, epoxy or a mixture thereof.

6. The method according to claim 1 wherein the step of forming the distal end portion includes rotating and machining the proximal core wire to form the distal end portion, and the step of forming the proximal end portion includes rotating and machining the distal core wire to form the proximal end portion.

7. The method according to claim 1 wherein the first variable diameter is configured to taper distally along a first partial length of the distal end portion and to flare distally along a second partial length of the distal end portion, the second partial length being distal to the first partial length, and wherein the second variable diameter is configured to taper proximally along a third partial length of the proximal end portion and to flare proximally along a fourth partial length of the proximal end portion, the fourth partial length being proximal to the third partial length.

8. The method according to claim 7 wherein the joining member extends over the first, second, third and fourth partial lengths, and the method further includes removing material from the proximal core wire to form a tapered section, the tapered section being proximal to the distal end portion and having a third variable diameter that is less than the maximum diameter of the proximal core wire but greater than the first variable diameter, the third variable diameter is configured to taper distally along a length of the tapered section.

9. A wire guide comprising:
a proximal core wire having a distal end portion, the distal end having a first variable diameter less than a maximum diameter of the proximal core wire, the first variable diameter is configured to flare distally along at least a partial length of the distal end portion
a distal core wire having a proximal end portion, the proximal end portion having a second variable diameter less than a maximum diameter of the distal core wire, the second variable diameter is configured to flare proximally along at least a partial length of the proximal end portion; and
a joining member formed of polymeric material overmolded onto the distal and proximal end portions, the joining member coupling the distal and proximal end portions together.

10. The wire guide according to claim 9 wherein the first variable diameter is configured to flare distally to form a first frustoconical section of the distal end portion, and the second variable diameter is configured to flare proximally to form a second frustoconical section of the proximal end portion.

11. The wire guide according to claim 9 wherein the proximal core wire comprises a first material and the distal core wire comprises a second material different than the first material, the first material being relatively stiffer than the second material.

12. The wire guide according to claim 9 wherein the first variable diameter is configured to taper distally along a first partial length of the distal end portion and to flare distally along a second partial length of the distal end portion, the second partial length being distal to the first partial length, and wherein the second variable diameter is configured to taper proximally along a third partial length of the proximal end portion and to flare proximally along a fourth partial length of the proximal end portion, the fourth partial length being proximal to the third partial length.

13. The wire guide according to claim 12 wherein the joining member extends over the first, second, third and fourth partial lengths, and the proximal core wire has a tapered section proximal to the distal end portion, the tapered section having a third variable diameter that is less than the maximum diameter of the proximal core wire but greater than the first variable diameter and is configured to taper distally along a length of the tapered section.

14. The wire guide according to claim 9 further comprising a collar distal to the proximal end portion disposed about the distal core wire.

15. A catheter kit comprising:
a guide catheter for insertion into a patient; and
a wire guide for providing the guide catheter a path during insertion into the patient, the wire guide comprising:
a proximal core wire having a distal end portion, the distal end having a first variable diameter less than a maximum diameter of the proximal core wire, the first variable diameter is configured to flare distally along at least a partial length of the distal end portion
distal core wire having a proximal end portion, the proximal end portion having a second variable diameter less than a maximum diameter of the distal core wire, the second variable diameter is configured to flare proximally along at least a partial length of the proximal end portion; and
a joining member formed of polymeric material overmolded onto the distal and proximal end portions, the joining member coupling the distal and proximal end portions together.
